(19) United States
(12) Patent Application Publication
(10) Pub. No.: US 2015/0231389 A1
(43) Pub. Date: Aug. 20, 2015

(54) SELECTIVE AUTONOMIC STIMULATION OF THE AV NODE FAT PAD TO CONTROL RAPID POST-OPERATIVE ATRIAL ARRHYTHMIAS

(71) Applicants: Marco A. MERCADER, Washington, DC (US); Jeffrey MOAK, Washington, DC (US)

(72) Inventors: Marco A. MERCADER, Washington, DC (US); Jeffrey MOAK, Washington, DC (US)

(21) Appl. No.: 14/700,698

(22) Filed: Apr. 30, 2015

Related U.S. Application Data
(63) Continuation-in-part of application No. PCT/US2013/068001, filed on Nov. 1, 2013.

(51) Int. Cl. A61N 1/05; A61N 1/365; A61B 5/042

(52) U.S. Cl. CPC A61N 1/059; A61B 5/0422; A61N 1/365

(57) ABSTRACT

Arrhythmia is a condition involving a rapid or slow heart rhythm that temporarily complicates the postoperative recovery from open heart surgery. Arrhythmias can be caused by sinus node dysfunction—slow heart rate or abnormalities in atrial conduction and automaticity that predispose to rapid heart rates. Current temporary treatments for rapid atrial arrhythmias are medications or external electrical cardioversion shocks. The present invention describes a method of treatment of postoperative arrhythmias and a device that includes a multipolar plaque electrode implanted on the atrioventricular (AV) node fat pad during the initial open heart surgery, leads that exit the body, an external controller connected to the leads that delivers an electrical stimulus, and a system to monitor the heart in order to optimize cardiac performance through the selection of individual stimulation poles and the stimulus parameters. The direct electrical stimulus to the AV node lowers the heart rate to achieve a “normal” heart rate or rhythm without medications or powerful shocks. The treatment is painless and without obvious side effects. The leads are designed to be removable or degradable once the patient is no longer at risk.

### RELATED APPLICATIONS

This application is a continuation-in-part of PCT/US2013/068001, filed Nov. 1, 2013, which claims the benefit of U.S. Provisional Application No. 61/721,334, filed Nov. 1, 2012. The entire content of those applications are incorporated herein by reference.

### FIELD OF THE INVENTION

The present invention relates to devices that treat dangerous rapid heart rates, or atrial arrhythmias, resulting during recovery from open heart surgery in both children and adult patients. The present invention is a temporary (removable) solution enabling painless delivery of electrical stimulation selectively to the AV node fat pad to the heart in order to optimize cardiac performance and reduce postoperative complications.

### BACKGROUND OF THE INVENTION

Open heart surgery patients are at risk of developing supraventricular arrhythmias including atrial tachycardia (AT), junctional ectopic tachycardia (JET), and atrial fibrillation (AF). Adult patients develop post-operative AF in approximately 30% of open heart surgeries. In children, post-operative atrial arrhythmias occur in 20-30% of patients (JET—10-15%, and AT—5-10%). These arrhythmias result in low cardiac output and hypotension, thus compromising recovery from surgery.

Currently, caregivers depend on the administration of intravenous medications or external cardioversion to control these post-operative arrhythmias. Medication administration can result in adverse effects, in some drug studies up to 50-75% patients have adverse effects. External cardioversion involves delivery of a powerful shock to a large area, which causes significant pain and necessitates application of general anaesthetic. Electrical cardioversion can result in depression of cardiac function, and need to administer medications to improve heart function.

The field of treatment for atrial arrhythmias also includes implantable devices. Because these devices are permanently implanted, they are not appropriate for patients who will transiently be at risk for atrial arrhythmias during recovery from open heart surgery. If an implantable device were used in such a case, a second surgery would be required to remove the implanted device. Such implantable devices incorporate both a ventricular pacemaker to maintain high enough heart rate and an atrial stimulator to decrease rapid atrial rates. An example is the device disclosed in U.S. Pat. No. 8,190,257 issued to Moffitt et al. This device decreases atrial rates by delivering electrical stimulation to the atrioventricular (AV) node fat pad.

A limitation of such implantable devices is the inclusion of only one electrode (with two pacing poles) for stimulation of the AV node fat pad. When attaching the electrode to the fat pad, it is possible that the first location selected will not provide adequate control of the atrial rhythm, requiring surgeons to continue to move the electrode to find an optimal site for the electrode position to enable control of the cardiac arrhythmia. Once in place, any inadvertent movement or alteration in the desired physiological responsiveness requires a second surgery to replace or reposition and secure the electrode.

Other general features of such implantable devices are a sensor, a controller, and a software system to detect atrial arrhythmias and determine what electrical stimulus to deliver, and when to deliver the stimulus to decrease the ventricular rate, during the atrial arrhythmia. A limitation of current implantable devices in particular is the lack of an automated system that can continuously monitor, detect and properly control JET. Currently no selective parasympathetic stimulation protocol is known for controlling JET. This limitation exists in part due to the lack of a good biological model system for studying JET and the resultant ability to create such a detection and control system.

### SUMMARY OF THE INVENTION

The present inventors established an animal model in pigs of human postoperative JET entitled 1) “Newly created animal model of human postoperative junctional ectopic tachycardia,” Moak et al., Journal of Thoracic and Cardiovascular Surgery, Sep. 26, 2012 (online publication) and 2) Nonpharmacologic control of postoperative supraventricular arrhythmias using AV nodal fat pad stimulation in a young animal open heart surgical model, to Moak J P, Mercader M A, He D, Trachiotis G, Langert J, Blicharz A, Montague E, Li X, Cheng McCarter R, Bornzin G A, Martin G R, Jonas R A, Circ Arrhythm Electrophysiol, 2013 June; 6(3):641-7, doi: 10.1161/CIRCEP.113.000090, Epub 2013 May 20. The entire content of those documents are hereby incorporated by reference.

Patients recovering from open heart surgeries need a new method of treatment for postoperative atrial arrhythmias that is temporary, painless, and effective. One object of the present invention is a method of treatment that utilizes a sensor to detect postoperative atrial arrhythmias and a catheter-like device with an electrode pad or “plaque” that is attached to the AV node fat pad to effect normalization of atrial rhythm or to slow the ventricular rate response to the atrial arrhythmia. In addition to the method of treatment, another object of the present invention describes a fully automated system that detects and responds to atrial arrhythmias resulting in a clinically desired correction of atrial rate and restoration of cardiac function. This system includes the implantable device, the external control instrumentation and the software system necessary to collect data regarding atrial arrhythmias and interpret such to determine appropriate corrective application of electrical stimulation, including selection of appropriate output parameters, and to maintain atrial rhythm within clinically acceptable performance criteria.

Another desirable object of the present invention is that the catheter-like device with an electrode pad or “plaque” is a multipole electrode with the characteristic that any combination of pair and polarity of poles can be selected to deliver an optimized current output thus eliminating the need for repositioning of the device at the time of surgery or in subsequent corrective surgeries in order to achieve the desired parasympathetic stimulation.

In one embodiment, the multipole plaque electrode remains sutured to the fat pad. After recovery, the leads are removed from the body without surgery. In another embodiment, the sutures and multipole electrode plaque are fully biodegradable, breaking down into components that are absorbed by the body, leaving only the electrode leads to be pulled.

### BRIEF DESCRIPTION OF THE DRAWINGS

FIG. 1 is a diagram of the system for the treatment of Atrial Arrhythmias in accordance with the present invention;

FIG. 2 shows the Multipole Electrode Plaque Assembly used in FIG. 1;

FIG. 3 is a plan view of the Multipole Electrode Catheter Assembly Positioning on the AV Node Fat Pad;

FIG. 4 shows signal diagrams of the AV Node Fat Pad Stimulation—Atrial Fibrillation and Complete AV Block;

FIG. 5 shows signal diagrams of Fat Pad Stimulation During Atrial Fibrillation;

FIG. 6 shows signal diagrams of AVN Fat Pad Stimulation: Conversion of Junctional Ectopic Tachycardia (JET) to Sinus Rhythm;

FIGS. 7 and 8 show mating connectors that provide the ability to separate the plaque electrode from the connecting cable;

FIG. 9 is a graph showing how increasing stimulation voltage results in higher reduction of heart rate; and

FIG. 10 is a graph showing maximum and minimum heart rate reduction during atrial fibrillation by selecting the most effective pair of electrodes to stimulate the AV node fat pad.

### DETAILED DESCRIPTION OF THE INVENTION

In describing a preferred embodiment of the invention illustrated in the drawings, specific terminology will be resorted to for the sake of clarity. However, the invention is not intended to be limited to the specific terms so selected, and it is to be understood that each specific term includes all technical equivalents that operate in similar manner to accomplish a similar purpose. Several preferred embodiments of the invention are described for illustrative purposes, it being understood that the invention may be embodied in other forms not specifically shown in the drawings.

The invention described herein is directed to a surgical device, system, and method for the treatment of atrial arrhythmias. As required, embodiments of the present invention are disclosed herein. However, the disclosed embodiments are merely exemplary, and it should be understood that the invention may be embodied in a number of various and alternative forms. The figures are not to scale, and some features may be exaggerated or minimized to show details of particular elements, while related elements may have been eliminated to prevent obscuring novel aspects. Therefore, specific structural and functional details disclosed herein are not to be interpreted as limiting, but merely as a basis for the claims, and as a representative basis for teaching one skilled in the art to variously employ the present invention. For purposes of teaching and not limitation, the illustrated embodiments are directed to a surgical device and associated system.

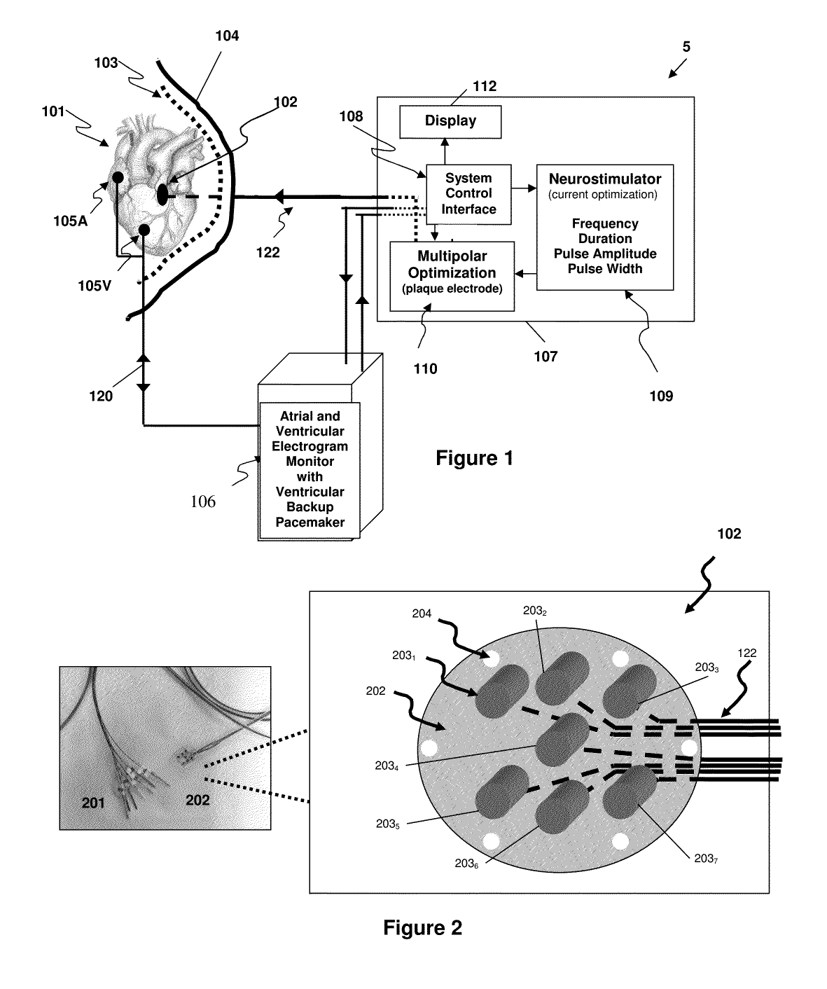

Referring now to the drawings, FIG. 1 shows a system 5 and method of treatment for the control of postoperative atrial arrhythmias. The system includes an electrode assembly 102 and a control instrumentation 107. The electrode assembly 102 is in communication with the control instrument 107. The multi-pole electrode assembly 102 or “plaque” is surgically attached to the AV node fat pad, and together with its associated instrumentation 107, effects stabilization of the heart rhythm and associated optimization of cardiac performance measurements (e.g., blood pressure) to within clinically defined and programmable parameters. Selective stimulation of the AV node fat pad is painless and has no obvious side effects.

The implantable electrode assembly 102 is used in open heart surgery and is designed to be surgically attached to the heart 101, more specifically to the AV node fat pad. The assembly 102 is preferably in the form of a catheter-like assembly 102. The electrode assembly 102 is shown implanted in a patient and sutured to the patient's heart 101. The implantable assembly 102 protrudes through the pericardium 103 and chest wall 104 of the patient after closure.

In the embodiment shown, two electrode pairs containing one or more sensors 105A, 105V are provided that detect abnormal atrial or ventricular rhythms. Any suitable sensors 105A, V can be used, such as those that detect atrial and/or ventricular arrhythmias (atrial and ventricular pacing wires sewn to the heart as a bipolar pair). The sensors 105A, V are connected to an associated control instrumentation or pacemaker 106 via leads 120. The pacemaker 106 receives the detected rhythms from the sensors 105A, V, interprets those rhythms as normal or abnormal. In addition, if the rhythms are too slow and pacing is required, the pacemaker 106 delivers a pacing current back to the ventricle via the lead 120 to the ventricular electrode 105V. The pacemaker 106 monitors atrial and/or ventricular arrhythmias, including JET.

The instrument 107 includes a cardiac performance display (monitor) 112, a system controller 108, neurostimulator 109, sensor electrode 105, and optimizer 110. The detected rhythms, as well as the status of the atrial and/or ventricular arrhythmias, is relayed from the pacemaker 106 to the system controller 108. The system controller 108 responds to detected abnormal atrial or ventricular activity and provides corrective stimulation of the AV node fat pad until optimal cardiac performance is achieved. The controller interface 108 is a microprocessor and associated software logic contains components that evaluate and modify multiple parameters. For instance, the controller 108 can (a) monitor atrial rate and timing in relationship to the ventricle, (b) monitor ventricular rate and timing in relationship to the atrium, (c) pre-programmed high and low heart rates above which and below which will trigger action by the system controller, (d) fat pad stimulation controller logic circuit to vary electrode pole combinations and polarity, pulse amplitude, pulse width and stimulation frequency, (e) cardiac rhythm diagnosis logic circuit, (f) demand ventricular pacing logic circuit, and (g) user modification input.

If cardiac performance is outside the limits of clinically defined appropriate “norms”, the system controller 108 communicates to the neurostimulator 109 within which a series of current optimization protocols are initiated to control frequency (Hz), duration (msec), intensity (mA) and pulse width modulation (msec) of the outbound current to the implanted multipole electrode plaque assembly 102. The controller 108 can be used to adjust the necessary variables for the delivery of current including the duration of stimulation, frequency, pulse width, and current strength (amplitude) of the fat pad stimulation, that is then generated by the neurostimulator 109. The controller 108 determines the type of arrhythmia using atrial and ventricular logic to detect atrial fibrillation, atrial tachycardia, junction ectopic tachycardia and ventricular tachycardia. The controller 108 analyzes the ventricular rate obtained from the ventricular sensor, either atrial 105A or ventricular 105V, to determine if triggering stimulation of the fat pad should take place. The system as a whole would maintain, as one example, a clinically desirable heart rate, keeping the heart rate between 60 bpm and 100 bpm, depending on patient age, hemodynamic state and programmable heart rate ranges that the bedside clinician deems necessary. In the event of a heart rate that is too slow, the controller 108 will provide back-up ventricular pacing at a predetermined rate, to the sensor 105V via lead 120 and pacemaker 106. Thus, the system detects the intrinsic heart rate using electrocardiogram electrodes and turns on/off stimulation if heart rate is above a certain rate.

Thus, the controller 108 causes the neurostimulator 109 to general an appropriate stimulation signal. The desired stimulations signal can be derived from a number of factors. For instance, as shown in Table 1, the voltage level of stimulation at the fat pad site will have a direct relationship to the change in the ventricular rate during the arrhythmia. A stimulation signal at 10 volts will change the ventricular rate by −48%; whereas a stimulation signal at 15 volts will change the ventricular rate by −56%. By changing either the site of pacing (Table 2, below), the stimulation voltage (Table 1, below), stimulation frequency (Hz), the system controller can optimize the heart rate within the desired programmed range and optimize the blood pressure recorded from an indwelling arterial line. Increasing the stimulation voltage at a constant site was shown in the example in Table 1 to further slow the ventricular rate during an episode of atrial fibrillation. Cardiac output will be calculated using the pulse contour method, such as discussed in Br J Anaesth. 2011 August; 107(2):202-8. doi: 10.1093/bja/aer123. Epub 2011 Jun. 10, Comparison between an uncalibrated pulse contour method and thermodilution technique for cardiac output estimation in septic patients, Franchi F, Silvestri R, Cubattoli L, Taccone F S, Donadello K, Romano S M, Giomarelli P, McBride W T, Scolletta S, the contents of which are hereby incorporated by reference. Optimization of stimulation parameters will be determined upon a desired ventricular heart rate, blood pressure and cardiac output.

The values can be set manually, or automatically determined by the controller 108.

TABLE 1
Fat Pad Stimulation during AFIB - Effect of Stimulation
Voltage on Ventricular Heart Rate

| Stimulation Voltage (volts) | % Change in Ventricular Rate |
|---|---|
| 10 | −48 |
| 12 | −49 |
| 15 | −56 |
| 18 | −61 |
| 20 | −69 |

In addition, as shown in FIG. 2, the plaque assembly 102 has a plurality of electrodes 203. The system control interface 108 interprets the efficacy of the outbound current stimulation pulses with regard to the restoration of normal rhythm. If necessary, the controller 108 optimizes the choice of electrode pole pairs 203 in the assembly 102 using a response optimizer 110.

That is, the controller 108 determines which of the plurality of poles 203 should receive the stimulation signal to stimulate the AV node fat pad. This may be based on the signals input from 105A, V. Illustrative data is shown in Table 2 below (ventricular heart rate response during atrial fibrillation using different electrode pole combinations. In Table 2, if electrode poles 203₁ and 203₅ are selected, the ventricular rate will change −31%; whereas if electrode poles 203₂ and 203₆ are selected, the ventricular rate will change −9%. Thus, depending on the correction to be made, a specific pole combination may be better than other pole combinations. This selection can be done manually by viewing the relevant rhythms on the display 112 and testing the results of the various pole combinations. Or can be done automatically through the controller 108. The system can perform an initialization test using different electrode pole combinations to determine the electrode pair that results in the largest effect, in the illustrative example, greatest slowing of the heart rate during the arrhythmia—electrodes 5 and 6. This combination will be used predominantly throughout the post-operative recovery period by the system to control cardiac performance. The system might recheck if these two pole combinations continue to be the optimal pair when stimulation parameters that are need to optimize cardiac performance exceed programmed thresholds.

TABLE 2
Fat Pad Stimulation during AFIB - Effect of Varying
Probe Pole Combination on Ventricular Heart Rate

| Probe Pole Combination | % Change in Ventricular Rate |
|---|---|
| 1-5 | −31 |
| 2-6 | −09 |
| 3-7 | +08 |
| 1-2 | −02 |
| 1-4 | −12 |
| 1-7 | −17 |
| 5-6 | −32 |
| 5-7 | −26 |
| 6-4 | −12 |

The controller 108 determines which poles to utilize based on the largest drop in heart rate from testing all possible pole combinations, as illustrated in Table 2. It then sends a control signal to the optimizer 110 to use those two poles. The optimizer 110 receives the stimulation signal output from the neurostimulator 109 and transmits the stimulation signal with the optimal current parameters, to the selected electrode pairs of the plaque assembly 102, via lead lines 122.

Cardiac performance optimization is monitored. If cardiac performance is within predefined clinical “norms”, for example keeping the heart rate between 60 bpm and 100 bpm, the current and electrode configurations are maintained. If performance is sub-optimal, the sequence of current and electrode configuration optimizations is repeated using the optimizer 110 until clinically acceptable cardiac performance measurements are obtained. In the event of a heart rate that is too slow, the system will provide back-up ventricular pacing via the backup pacemaker 106. All patient cardiac performance results as well as current and electrode configurations are displayed on a monitor 112.

In an alternate embodiment of the invention, the atrial and/or ventricular monitoring sensor 105, including the backup ventricular pacing function of the pacemaker 106 could be integrated into the system control and modulation instrumentation 107. In either case, the system control and modulation instrumentation 107 can also accept inputs from conventional cardio-hemodynamic monitoring devices (e.g., heart rate, blood pressure, left atrial pressure, pulmonary artery pressure, dP/dT) as desired to give a complete clinical view of cardiac performance. Thus, while the monitor/pacemaker 106 is shown as a separate element than the instrument 107, it will be appreciated that the pacemaker 106 can be integrated with the instrument 107, and the operation of the pacemaker 106 can be integrated with or performed by the system controller 108.

The leads 122 extend from the plaque assembly 102 inside the body to the instrumentation 107 is outside the body. The leads 122 can be removed without surgery after a patient has recovered from the initial open heart surgery. Because the leads exit the chest wall, the control instrumentation 107 and pacemaker 106 are external. This allows for greater functionality of the pacemaker 106 and the control device 107, saves space inside the body and therefore is less invasive.

FIG. 2 shows the implanted assembly 102 of FIG. 1 in further detail. The implantable device 102 includes multi-pole electrode leads 122 and a multi-pole non-tissue penetrating electrode assembly 202 together in a multifilar cable. The electrode head assembly or “plaque” 202 has two or more individually addressable electrodes or poles 203 and various suture holes 204 that allow for surgical discretion of electrode placement. In the present illustrative and non-limiting embodiment, the plaque 202 is thin and flat plate with blunt edges and is curved (here with an oval shape). The suture holes 204 are positioned just inside the outer circumferential edges of the plaque 202, so that the plaque 202 can be sutured in place to the patient's heart.

The plate has a first planar side that has a surface which faces toward the heart when the plaque 202 is sewn thereto, and a second planar side opposite the first side that has a surface which faces away from the heart when the plaque 202 is sewn thereto. The electrodes 203 are positioned provide an electrical output at the first side of said plate and said plurality of leads are each removably coupled with a respective one of said plurality of electrodes via the second side of said plate. Thus, the poles 203 are in direct contact with the heart when the plaque 202 is sutured to the heart, so that the poles 203 can sense the heart rhythms and also impart the stimulation signal to the heart. And the leads 122 pass on the opposite side of the plate so that they do not interfere with the poles 203 or otherwise come between and create a gap between the first side of the plate and the heart.

The plaque 202 is made of a plastic, such as a polyethelene, so it is electrically insulated and does not interfere with the operation of the electrodes 203 or leads 122. The plaque 202 is flexible so that it can bend with the motion of the heart and not injure any cardiac structure. The electrodes 203 can be affixed to the plaque 202 in any suitable manner, such as by adhesive or a fastener. Each electrode in 1 mm in length and circumference. The oval plague has a dimension of 8 mm by 6 mm. The electrodes 203 can be used to record an atrial and ventricular signal and to impart the stimulation signal to the AV fat pad node to correct for AV, AT or JET. The multipole electrode plaque 202 is shown with seven (7) individual electrode poles. Selection of any two (2) of the electrode poles can provide the desirable vector and choice of polarity for stimulation. This allows for a wide range of possible combinations of vectors for stimulation.

In the embodiment shown, the poles 203 are oblong in shape with curved ends, non-tissue penetrating so as to avoid tissue injury and deep atrial stimulation. Three poles 203 are located at one longitudinal side of the plaque 202, three poles 203 are located at the opposite longitudinal side of the plaque 202, and one pole 203 is located in the middle of the plaque 202. There are an equal number of leads 122 as poles 203, so that each lead 122 connects to a respective one of the pole 203. However, it is appreciated that a single lead 122 can connect to more than one pole 203. And, a lead 122 need not be connected to every pole 203 depending on the application. The pole combinations allow for a broad choice of interelectrode spacing during bipolar stimulation (2 mm interelectrode spacing, 5 mm spacing and 10 mm spacing).

In another embodiment of the invention, the plaque can have conductive pathways extending inside the plaque 202. A separate conductive pathway extends from each electrode 203 and all the pathways come together (though remain separate) at one end of the plaque 202, as shown in FIG. 2. At that point, a detachable connector 210 (FIG. 7) can be provided that is fixedly mounted to the plaque 202. The plaque connector 210 can have female receptacles, with each receptacle connecting to one of the electrodes 203 along a respective pathway. In addition, the leads 122 can come together as a mating connector 212 (FIG. 8). The mating connector 212 has male receptacles, with each male receptacle connecting to one of the leads 122. The female and male connectors 210, 212 mate and engage each other in a removable fashion. Preferably, the female connector 210 is on the plaque 202 so that the male prongs do not remain inside the patient after the leads 122 are withdrawn. In addition, the female connector 210 can slide into the male connector 212 to provide a reliable connection.

Accordingly, a surgeon can suture the plaque 202 in place, then connect the connectors 210, 212. Once the temporary treatment is complete, the physician can pull on the leads 122 to detach the connectors 210, 212 from one another and remove the leads 122 from the patient. The multipole electrode plaque 102 will remain sutured to the fat pad. The ability to remove most of the apparatus 102 enables a temporary solution to a temporary period of high risk for atrial arrhythmias, while providing the painless and effective selective stimulation that could otherwise be achieved with a permanent implantable device. The lead lines 120 for the sensors 105A, V can be removed in a similar manner. In another embodiment the sutures and multipole plaque electrode 102 are fully biodegradable, breaking down into components that are absorbed by the body by being made of any suitable material, such as the one discussed in Huang et al., A Physically Transient Form of Silicon Electronics, Science 337:6102 pp. 1640-1644, Sep. 28, 2012, the entire contents of which are hereby incorporated by reference.

The general design of the size of the plaque 202, its electrode poles 203, configurability and means of attachment is to minimize atrial tissue stimulation. The material comprising the plaque 202 may be of any one of a number of conventional surgical materials or the plaque may be comprised completely of any surgically suitable biodegradable material. The materials in these embodiments are not limiting.

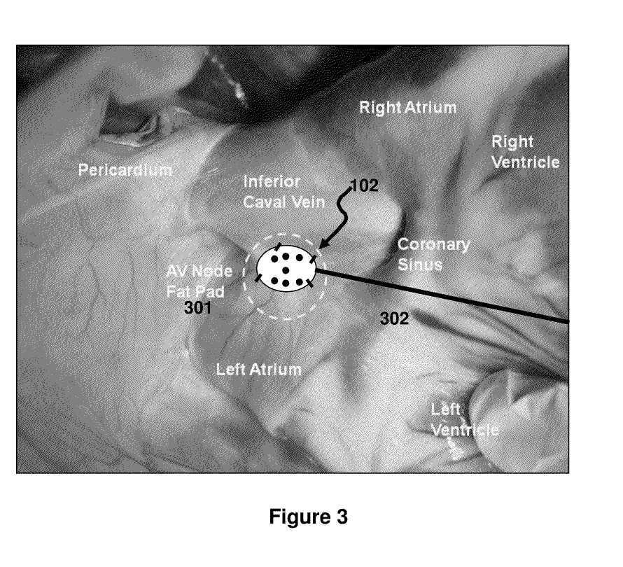

FIG. 3 illustrates the positioning of the multipole selectable electrode plaque assembly 102 on the AV Node Fat Pad 301, where the atrium 301 and ventricle 302 are shown. The multipolar selectable electrode plaque 102 eliminates the need for repositioning of the device at the time of surgery in order to achieve parasympathetic stimulation. The custom-made multipolar electrode plaque is sutured onto the AV node fat pad located at the junction of the inferior vena cava and left atrium 301. Any combination of pair and polarity of poles can be selected to deliver an optimized current at any one or more of the poles 203, thus eliminating the need for repositioning of the device at the time of surgery or in subsequent corrective surgeries in order to achieve the desired parasympathetic stimulation.

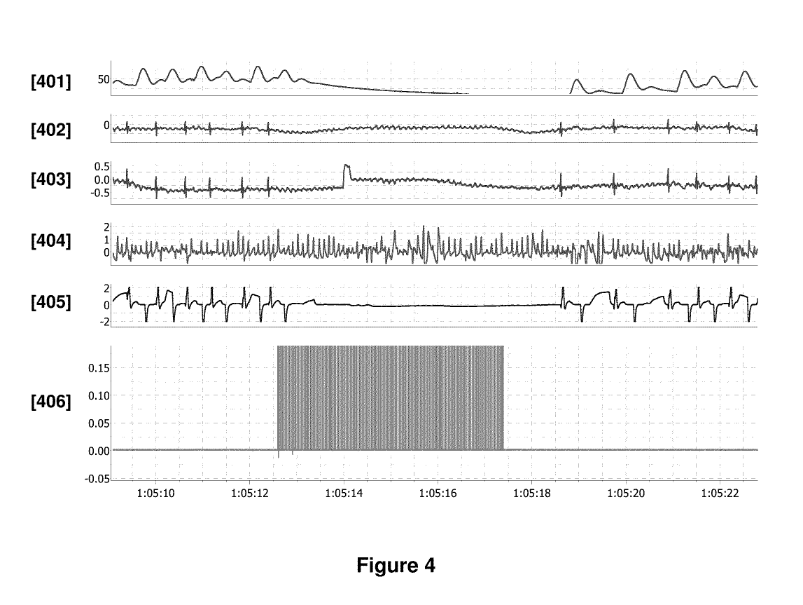

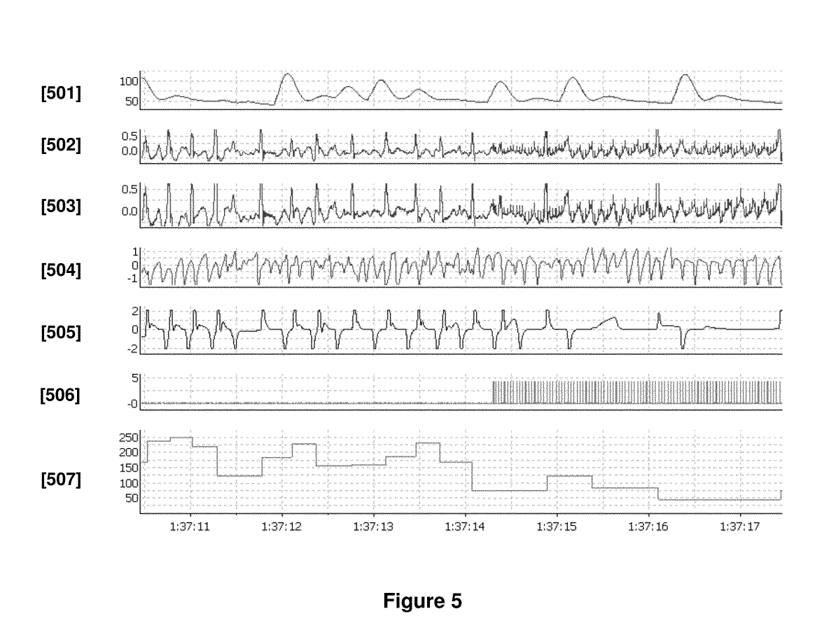

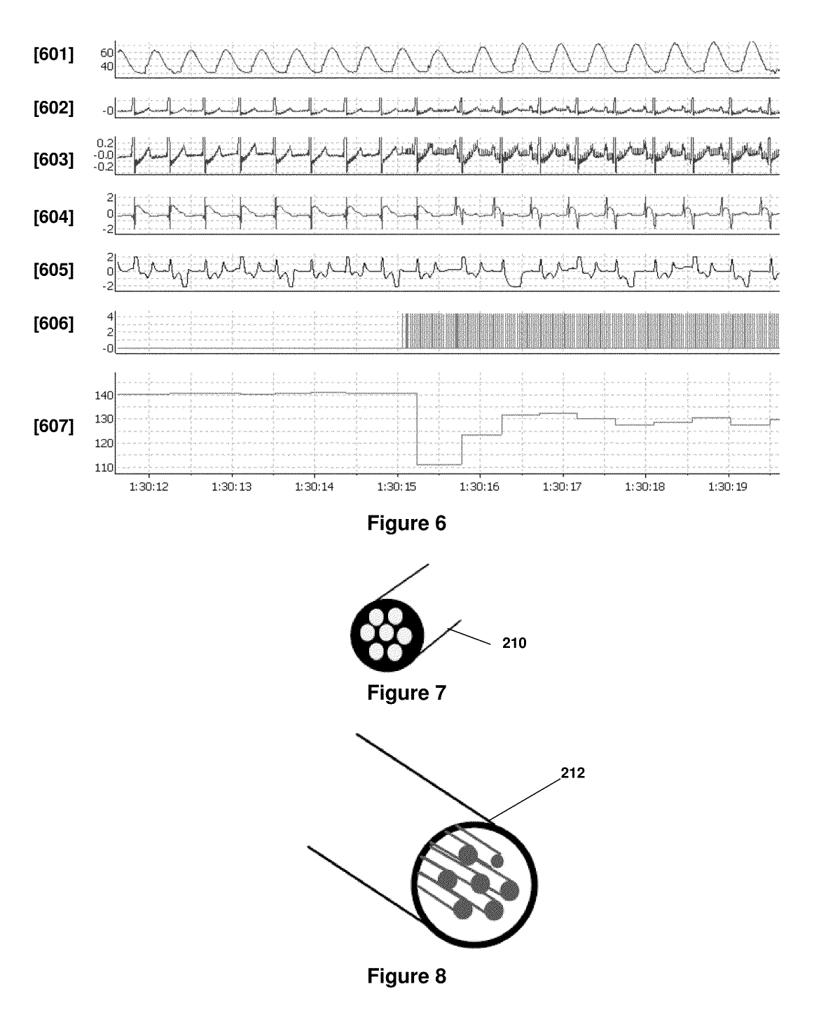

In addition to the implanted assembly 102, the present invention provides a novel stimulation signal for the treatment of all three forms of atrial arrhythmias including atrial tachycardia, junctional ectopic tachycardia (JET), and atrial fibrillation. The stimulation signal is a square wave pulse. The figures demonstrate the effect of fat pad stimulation on control of atrial fibrillation (FIGS. 4 and 5) and JET (FIG. 6). It should be appreciated that although the implanted assembly device 102 and the stimulation signals (device 107) are described as being utilized together, each has its own separate utility and need not be used with the other. For instance, the stimulation signal can be utilized without the implanted assembly 102, such as with other implantable devices. And the implanted assembly 102 can be used to apply other stimulation signals other than the ones described here.

The protocol for controlling JET, AT and AF is embodied within and implemented by the neurostimulator 109 as modified by the controller 108 of the present invention. In one embodiment, the method involves or software encodes for parasympathetic nerve stimulation via the AV node fat pad that is continuous up to 20 seconds in duration, with a pulse width of 0.10-0.15 milliseconds, frequency of 20-50 hertz, and current strength of 40 milliamps (voltage 5-20 volts). The stimulation has an effect on the AV node and therefore controls all supraventricular arrhythmias (JET, AT, AF). Fat pad stimulation is performed at varying frequencies, and outputs to construct a stimulus-response relationship using AV node block as the end point. This is important to select the appropriate frequency and current strength to be used on the particular patient being treated, see Table 2 above.

The AV node Wenckebach cycle length and effective refractory period is determined in the baseline state and during fad pad stimulation. This gives clinical feedback to the physicians on how effective the fat pad stimulation is for the particular patient being treated. As the instrument 107 applies a stimulation signal to the AV fat pad, the sensors 105A, V continue to detect the heart rhythms and apply pacing corrections as well as to adjust the stimulation signal being applied to the patient. The Wenckebach cycle length is discussed in Nodal recovery, dual pathway physiology, and concealed conduction determine complex AV dynamics in human atrial tachyarrhythmias, to M. Masse et al., Am J. Physiol 303, H1219-1228, Sep. 14, 2012, the content of which is hereby incorporated by reference.

FIGS. 4-6 show the stimulation signal for treatment during AF (FIGS. 4 and 5), and JET (FIG. 6), whereby the AV fat pad stimulation signals 406, 506 (FIGS. 4, 5) treat AF, and the AV fat pad stimulation signal 606 (FIG. 6) treats JET. Referring to FIG. 4, AV node fat pad stimulation signal 406 is shown in a canine model to alter AF 404 and can accomplish complete AV block 405. Commensurate attenuation of blood pressure 401 (mm Hg), electrocardiogram (ECG) activity at leads II 402 and III 403 are observable. Signal 404 is the atrial EGM recording, signal 405 is the ventricular EGM recording, and signal 406 is the Fat Pad Neurostimulation channel. Prior to neurostimulation 406 of the Fat Pat the cardiac rhythm, signal 404, is atrial fibrillation with a rapid ventricular response (approx. 500 msec). During intense neurostimulation 406, complete (third degree) AV block results, signal 404. The effect of neurostimulation is rapid and is achieved within less than 500 msec. After neurostimulation is stopped, signal 406, AV nodal conduction resumes with a graded response to the atrial fibrillation (gradual acceleration of the ventricular rate), signal 404.

Referring to FIG. 5, stimulation of the AV node fat pad 506 in a canine model alters AF in the atrial EGM 504 as evidenced in either ECG lead II 502 or III 503, and the atrial electrogram channel 504. Blood pressure (mm Hg) 501 and ventricular rate (bpm) 507 are also shown. The atrial electrogram 504 revealed continuous and fragmented electrical activity consistent with atrial fibrillation. Evident on ECG lead II 502 and II 503 was a rapid and irregular ventricular response in the ventricular EGM 505 to the atrial fibrillation. Following AV nodal fat pad stimulation 506, the ventricular rate significantly slows from approximately 200 to 75 beats/min (shown in signal 507). Accompanying electrical slowing of the ventricular rate was correction of the electrical-mechanical pulse deficit. In contrast when fat pad stimulation is terminated, the ventricular rate increases back to baseline. No change in the atrial electrical activity is evident on the atrial electrogram channel either before, during or after Fat Pad stimulation. In contrast to FIG. 4, neurostimulation of the AV nodal fat pad is less intense, thereby resulting in second degree AV block instead of third or complete AV block. The effect of AV nodal fat pad stimulation is confined to the AV nodal conduction, and has no effect on the atrial electrical activity.

Referring to FIG. 6, AV node fat pad stimulation 606 in a canine model demonstrates slowing of JET to lower right atrial rhythm 604. During AV nodal fat pad stimulation 606, evident in the figures is conversion of JET to sinus rhythm. If one focuses their attention to either ECG lead II 602 or III 603 and the atrial electrogram channel of the atrial EGM 604, the observer can see on the left hand side of the atrial electrogram channel 604, the atrial 604 and ventricular 605 electrograms occurred simultaneously, consistent with JET. Following fat pad stimulation 606, the ventricular rate slowed, and the atrial 604 and ventricular electrograms 605 separate, with the atrial electrogram 604 preceding the ventricular electrogram 605, indicating the development of sinus rhythm, i.e. the transition of JET to sinus rhythm. An increase in the systolic blood pressure (mm Hg) 601 and a decrease in ventricular rate (bpm) 607 are also observable accompanying the development of sinus rhythm. Fat pad stimulation slows the junctional rate during parasympathetic stimulation below the atrial rate, allowing the sinus node pacemaker to dominate the cardiac rhythm.

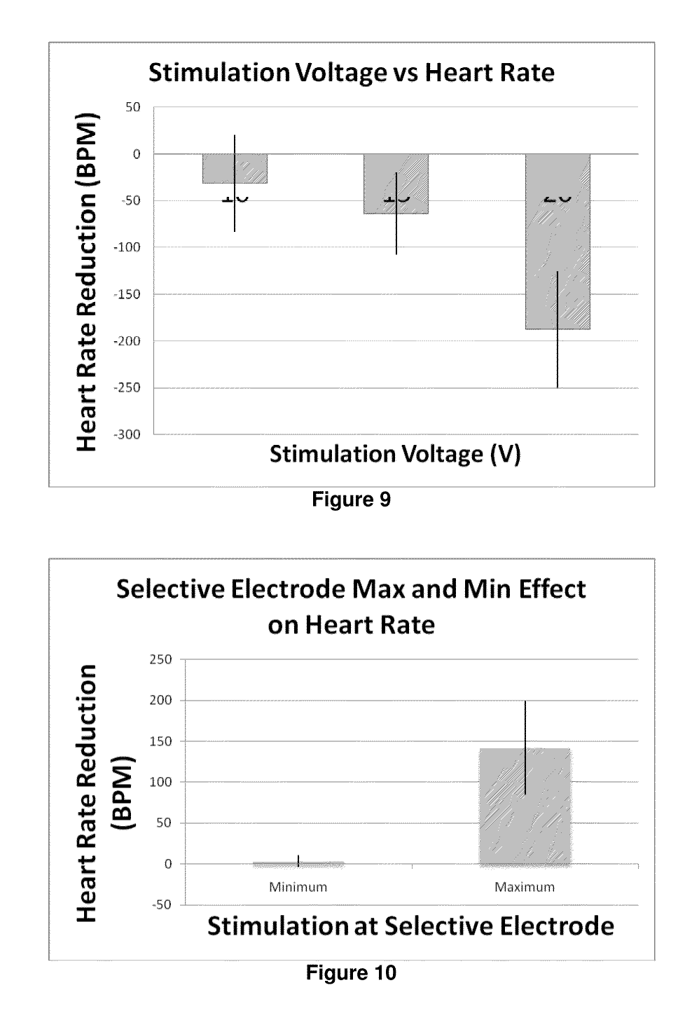

Our experiments have confirmed that the inferior right FP was more effective in slowing the ventricular rate response to atrial fibrillation in six animal experiments (−0.43±0.18% vs. −0.18±0.11%, p=0.03) and JET (−0.16±0.06 vs. 0.0±0.0, p=0.06). Selective site stimulation within a Fat Pad region could augment the effect of stimulation during AF (−0.48±0.21 (maximum effect) vs. 0.0±0.0% (least effect), p=0.01). Fat pad stimulation at increasing Voltage demonstrated a voltage-dependent effect (−0.12±0.19 (low V) vs. −0.63±0.21 (high V) %, p=0.01). FIG. 9 shows a summary of 5 experiments illustrating the heart rate reduction as a function of voltage applied to the AV node fat pad during AF. FIG. 10 shows a summary of 5 experiments illustrating the heart rate reduction following selective site stimulation (using the best pair of electrodes from the multiple electrodes available on the flat plaque electrode) of the inferior right fat pad during AF.

Based on our selective site stimulation studies, we have concluded that selective site stimulation within a fat pad region and optimizing the voltage output enhanced the efficacy of ventricular rate (VR) slowing during post-operative atrial arrhythmias. The multipolar electrode design allows for choosing among multiple sites within the inferior right fat pad allowing for the maximal slowing effect on VR. Thus, if the first pair of electrodes selected among the plurality of electrodes on the flat plate during surgery fails to work or looses its effect, a different second pair of electrodes can be selected among the plurality of electrodes after closing of the patient's chest wall without having to re-open the patient's chest and reposition the electrode. The inferior right fat pad (AV node fat pad) was more effective in decreasing VR during AF, suggesting its role in control of AV node conduction. The anterior right fat pad had little effect on JET compared with the IR fat pad, suggesting differences in mechanism of action between the two regions. The electrode catheter and pacing device can be used in humans for rate control of JET and AF in the near future.

In summary, AV node fat pad stimulation had a selective effect on the AV node decreasing AV nodal conduction, with little effect on atrial activity. No significant change in the sinus cycle length was observed, thereby showing that the effect is selective to the AV node and AV conduction. AV nodal fat pad stimulation slowed the ventricular rate during post-operative JET and atrial fibrillation in our young canine OHS model. These results demonstrate the ability of AV nodal fat pad stimulation to not only decrease AV nodal conduction during atrial fibrillation, but also peri-AV nodal automaticity during JET. On average, AV nodal FP stimulation significantly slowed the average ventricular rate during JET from 148±31 to 106±32 beats per minute in seven canine experiments (N=7; P<0.001). Sinus rhythm occurred in 7 of 7 experiments. When FP stimulation was terminated, the rate of JET gradually increased back to the baseline rate. Thus, while the stimulation signal is described for use with the plaque assembly 102, they each can be used separately. For instance, the stimulation signal can be utilized with any electrode configuration and the plaque assembly 102 can be utilized with any suitable stimulation signal and need not be applied to the AV node fat pad.

It is further noted that the instrumentation 107, and particularly the controller 108 and/or pacemaker 106, can be implemented by a computer or computing device having a processor to perform various functions and operations in accordance with the invention. The computer can be, for instance, a personal computer (PC) or server. In addition to the processor, the computer hardware may include one or more of a wide variety of components or subsystems including, for example, input devices, display 112, and a memory or storage device such as a database. All or parts of the system and processes can be implemented at the processor by software or other machine executable instructions which is stored on or read from computer-readable media for performing the processes described above. Computer readable media may include, for instance, hard disks, floppy disks, CD-ROM or other forms of computer-readable memory such as read-only memory (ROM) or random-access memory (RAM), solid-state, analog or other memories; optical and/or magnetic media; a centralized or distributed database; and/or caches.

The foregoing description and drawings should be considered as illustrative only of the principles of the invention. The invention may be configured in a variety of shapes and sizes and is not intended to be limited by the preferred embodiment. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention.

1. An electrode assembly for treatment of arrhythmias, the assembly comprising:
a substantially flat plate;
a plurality of electrodes coupled to said plate and configured to provide an electrical output;
a plurality of leads each removably connected with a respective one of said plurality of electrodes; and
at least one opening in said plate each configured to receive a suture so that said plate can be attached to an atrioventricular node fat pad on the epicardial surface of a heart during open heart surgery when the patient's chest wall is open, wherein a first pair of electrodes of the plurality of electrodes can be selected during surgery to provide the electrical output and a second pair of electrodes different than the first pair of electrodes can be selected after surgery when the patient's chest wall is closed to provide the electrical output.

2. The assembly of claim 1, wherein said plate is configured to be attached by the suture to an atrioventricular node fat pad on the epicardial surface of the heart, in a patient's body during open heart surgery.

3. The assembly of claim 1, wherein said plurality of leads are configured to be removed without surgery from the patient's body following the open heart surgery.

4. The assembly of claim 1, wherein said plurality of electrodes is comprised of more than two electrodes.

5. The assembly of claim 4, wherein a stimulation signal is applied to a selected pair within the plurality of electrodes allowing flexibility in configuring of the vector of stimulation using different bipolar configurations.

6. The assembly of claim 1, wherein said plate has a first planar side and a second planar side opposite the first side, with said plurality of electrodes configured to provide an electrical output at the first side of said plate and said plurality of leads are each removably coupled with a respective one of said plurality of electrodes via the second side of said plate.

7. The assembly of claim 1, further comprising an electrocardiogram lead sensor to detect a heart rhythm, said electrocardiogram lead sensor is external to the patient's body and coupled to a processor configured to detect an abnormal rhythm and rate based signals detected by the electrocardiogram lead sensor.

8. The assembly of claim 7, further comprising a cardiac performance monitor connected to said plurality of leads and located external to the patient's body, said cardiac performance monitor configured to determine if the heart rhythm is abnormal and based on the determination apply an electric current having a controlled frequency, duration, intensity and pulse width modulation to said plurality of electrodes via said plurality of leads.

9. The assembly of claim 1, further comprising a cardiac performance monitor located external to a patient's body, said cardiac performance monitor providing a current signal to selected ones of said plurality of electrodes fewer than all of said plurality of electrodes.

10. The assembly of claim 1, wherein said plate and said plurality of electrodes are biodegradable.

11. The assembly of claim 1, wherein the treatment includes AV node fat pad stimulation to control supraventricular arrhythmias including junctional ectopic tachycardia and atrial fibrillation.

12. The assembly of claim 1, wherein each of the plurality of leads has a proximal section with a proximal end attached to each one of the respective electrodes, a middle section removably attached to a connector which is coupled to a respective one of said plurality of electrodes and a distal section having a distal end attached to pins removably attached to a neuorostimulator.

13. The assembly of claim 1, further comprising a stimulation device configured to transmit a stimulation signal to an atrioventricular node fat pad on the epicardial surface of the heart via at least one of said plurality of electrodes.

14. The assembly of claim 13, wherein the stimulation signal treats junctional ectopic tachycardia.

15. The assembly of claim 13, wherein the stimulation signal treats atrial tachycardia and atrial fibrillation.

16. A method for controlling heart rate of arrhythmias, the method comprising:
attaching a substantially flat plate to the epicardial surface of the heart of a patient's body in the vicinity of the AV node fat pad using suture material through two available pin holes on the flat plate, the plate having a plurality of electrodes;
attaching a plurality of leads having a proximal end to a respective one of the plurality of electrodes;
removably attaching a distal end of each of the plurality of leads to pins to a processor located external to the patient's body;
setting a current provided by the processor to a desired width, frequency, duration, current, pulse repetition interval and pulses per trigger; and,
stimulating by applying the current through two of the plurality of electrodes.

17. The method of claim 16, wherein the leads each have a proximal section with a proximal end attached to each one of the respective electrodes, a middle section being removably attached to a connector which is coupled to a respective one of said plurality of electrodes and connecting a distal section of each of the leads having a distal end to pins removably attached to a neuorostimulator.

18. The method of claim 16, further comprising suturing the plurality of leads to the rib cage and passing distal end to outside of the patient's body and coupling two of the electrodes pins on the distal end to the processor.

19. The method of claim 16, further comprising detecting intrinsic heart rate using electrocardiogram electrodes and turning on / off stimulation if heart rate is above a certain rate.

20. An electrode assembly for treatment of arrhythmias, the assembly comprising:
a plate configured to be attached to the atrioventricular node fat pad on the epicardial surface of a patient's heart;
a plurality of electrodes coupled to said plate and configured to provide an electrical output; and
a controller configured to select a first pair of electrodes from the plurality of electrodes to provide a first stimulation signal to the patient's heart, and a second pair of electrodes from the plurality of electrodes to provide a second stimulation signal to the patient's heart, wherein the first pair of electrodes is different from the second pair of electrodes.

21. The assembly of claim 20, further comprising an optimizer configured to determine a first reduction in heart rate for the first pair of electrodes and a second reduction in heart rate for the second pair of electrodes, wherein said controller selects the first pair of electrodes or second pair of electrodes based on the determined first and second reductions of heart rate.

22. The assembly of claim 21, wherein said controller selects the first pair of electrodes during surgery and the second pair of electrodes after closing of the patient's chest wall without having to re-open the patient's chest or reposition the electrodes.

23. The assembly of claim 21, wherein said controller selects the first pair of electrodes if the first reduction is greater than the second reduction in heart rate, and selects the second pair of electrodes if the second reduction in heart rate is greater than the first reduction in heart rate.

24. The assembly of claim 20, wherein the plate is attached during open heart surgery when the patient's chest wall is open.
